(19) United States
(12) Patent Application Publication
(10) Pub. No.: US 2015/0099961 A1
(43) Pub. Date: Apr. 9, 2015

(54) PHOTO-ACOUSTIC PROBE FOR CLINICAL IMAGE

(71) Applicant: Electronics and Telecommunications Research Institute, Daejeon (KR)

(72) Inventors: Yong Jae LEE, Daejeon (KR); Bong Kyu KIM, Daejeon (KR); Hyun Woo SONG, Daejeon (KR); Chang Geun AHN, Daejeon (KR); Eun Ju JEONG, Daejeon (KR); Hyung Wook NOH, Daejeon (KR); Won Ick JANG, Daejeon (KR)

(73) Assignee: Electronics and Telecommunications Research Institute, Daejeon (KR)

(21) Appl. No.: 14/500,741

(22) Filed: Sep. 29, 2014

(30) Foreign Application Priority Data
Oct. 8, 2013 (KR) 10-2013-0119893
Apr. 30, 2014 (KR) 10-2014-0052405

(51) Int. Cl. A61B 5/00

(52) U.S. Cl. CPC A61B 5/0095
USPC 600/407

(57) ABSTRACT

Provided is a miniaturized photo-acoustic probe for a clinical image capable of effectively measuring a photo-acoustic signal by making an ultrasonic axis and an optical axis parallel. The photo-acoustic probe for a clinical image includes a laser generator configured to generate a laser beam, an ultrasound transducer disposed to be parallel to the laser generator and configured to analyze ultrasound output from an object, first and second reflectors configured to receive ultrasound generated in an axis identical to that of the laser beam generated by the laser generator, and a medium material configured to allow the laser to be transmitted from the first reflector to the object and increase ultrasound reflectivity of the first and the second reflector.

### CROSS-REFERENCE TO RELATED APPLICATIONS

This application claims priority under 35 U.S.C. §119 to Korean Patent Application No. 10-2013-0119893, filed on Oct. 08, 2013 and Korean Patent Application No. 10-2014-0052404, filed on Apr. 30, 2014, the disclosure of which is incorporated herein by reference in its entirety.

### TECHNICAL FIELD

The present invention relates to a photo-acoustic probe for a clinical image, and more particularly, to a miniaturized photo-acoustic probe for a clinical image capable of effectively measuring a photo-acoustic signal by coaxially aligning an ultrasonic axis and an optical axis.

### BACKGROUND

In general, photo-acoustic imaging techniques obtain information by generating a sound by light. General photo-acoustic imaging techniques will be described briefly with reference to FIG. 1.

FIG. 1 is a view illustrating a structure of a related art photo-acoustic probe.

As illustrated in FIG. 1, a related art photo-acoustic probe has a structure in which an ultrasound transducer 12 is disposed slopingly at a predetermined angle with respect to a pulse laser 11 to make an axis of a laser beam output from the pulse laser 11 and an axis of ultrasound incident to the ultrasound transducer 12 oblique to reduce a device between a path of a laser beam and a path of ultrasound as possible to minimize loss generated therebetween.

However, when the two paths are sloped at a predetermined angle, a dark zone may appear in a region 4 of FIG. 1, where an image cannot be measured. Also, the oblique structure may increase a volume due to a structural limitation causing user inconvenience.

Alternatively, an effective probe structure in which a glass plate is used to make a laser beam irradiated to a sample and an ultrasound beam placed coaxially to reduce a dark zone has been proposed.

However, since a connector inputting light to a probe and an ultrasound transducer that receives ultrasound is at a right angle (90°), it is difficult to use the probe structure.

### SUMMARY

Accordingly, the present invention provides a photo-acoustic probe for a clinical image capable of effectively measuring a photo-acoustic signal and miniaturizing a probe structure by adjusting an ultrasonic axis and an optical axis such to be coaxially placed.

In one general aspect, a photo-acoustic probe for a clinical image may include: a laser generator configured to generate a laser beam; an ultrasound transducer disposed to be parallel to the laser generator and configured to analyze ultrasound output from an object; first and second reflectors configured to receive ultrasound generated in an axis identical to that of the laser beam generated by the laser generator; and a medium material configured to allow the laser to be transmitted from the first reflector to the object and increase ultrasound reflectivity of the first and the second reflector.

The medium material may include a frame having an inner space and a liquid accommodated in the inner space. The first and second reflectors may be formed of a glass plate (slide glass).

The photo-acoustic probe may further include a lens and beam controller positioned between the laser generator and the medium material.

In another general aspect, a photo-acoustic probe for a clinical image may include: a laser generator configured to generate a laser beam; an ultrasound transducer disposed to be parallel to the laser generator and configured to analyze ultrasound output from an object; a first reflector configured to reflect the laser beam so as to be incident to the object; a second reflector configured to allow the laser beam reflected from the first reflector to be transmitted therethrough and reflect the ultrasound so as to be incident to the ultrasound transducer; and a medium material configured to transmit the laser beam to the object and transmit the generated ultrasound to the ultrasound transducer.

The medium material may include a frame having an inner space and a liquid accommodated in the inner space.

The first reflector may include a mirror or a prism, and the second reflector may include a glass plate (slide glass).

The second reflector may allow the laser beam to be transmitted therethrough and totally reflects the ultrasound.

The photo-acoustic probe may further include a lens and beam controller positioned between the laser generator and the medium material.

The laser generator and the ultrasound transducer may be disposed to be perpendicular to the laser beam incident to the object.

The laser generator and the ultrasound transducer may be disposed at a predetermined angle with respect to the laser beam incident to the object, rather than being perpendicular to the laser beam.

### BRIEF DESCRIPTION OF THE DRAWINGS

FIG. 1 is a view illustrating the related art structure of a photo-acoustic probe;

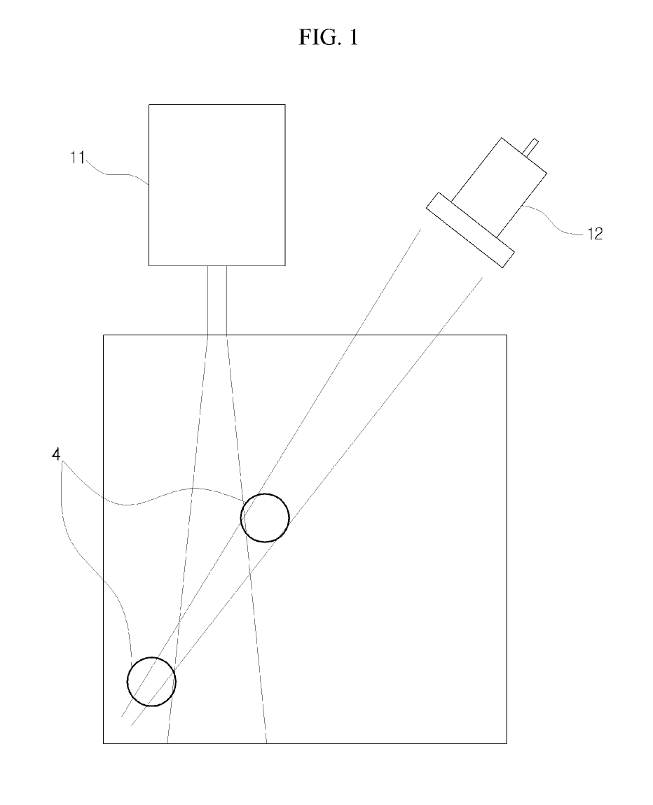

FIG. 2 is a view illustrating a structure of a photo-acoustic probe according to a first embodiment of the present invention;

FIG. 3 is a view illustrating a structure of a photo-acoustic probe according to a second embodiment of the present invention; and

FIG. 4 is a view illustrating a modified structure of the photo-acoustic probe illustrated in FIG. 3.

### DETAILED DESCRIPTION OF EMBODIMENTS

Hereinafter, embodiments of the present invention will be described in detail with reference to the accompanying drawings.

The advantages, features and aspects of the present invention will become apparent from the following description of the embodiments with reference to the accompanying drawings, which is set forth hereinafter. The present invention may, however, be embodied in different forms and should not be construed as limited to the embodiments set forth herein. Rather, these embodiments are provided so that this disclosure will be thorough and complete, and will fully convey the scope of the present invention to those skilled in the art. The terms used herein are for the purpose of describing particular embodiments only and are not intended to be limiting of example embodiments. As used herein, the singular forms “a,” “an” and “the” are intended to include the plural forms as well, unless the context clearly indicates otherwise.

Hereinafter, exemplary embodiments of the present invention will be described in detail with reference to the accompanying drawings. In adding reference numerals for elements in each figure, it should be noted that like reference numerals already used to denote like elements in other figures are used for elements wherever possible. Moreover, detailed descriptions related to well-known functions or configurations will be ruled out in order not to unnecessarily obscure subject matters of the present invention.

The present invention relates to a probe for effectively receiving ultrasound in a photo-acoustic imaging technology, a medical imaging technology. Specifically, the present invention provides a probe appropriate for effectively obtaining a photo-acoustic signal using a light source and an ultrasound transducer, which are used in the same manner as those of an existing method, but by aligning the light source and the ultrasound transducer such that axes thereof are parallel.

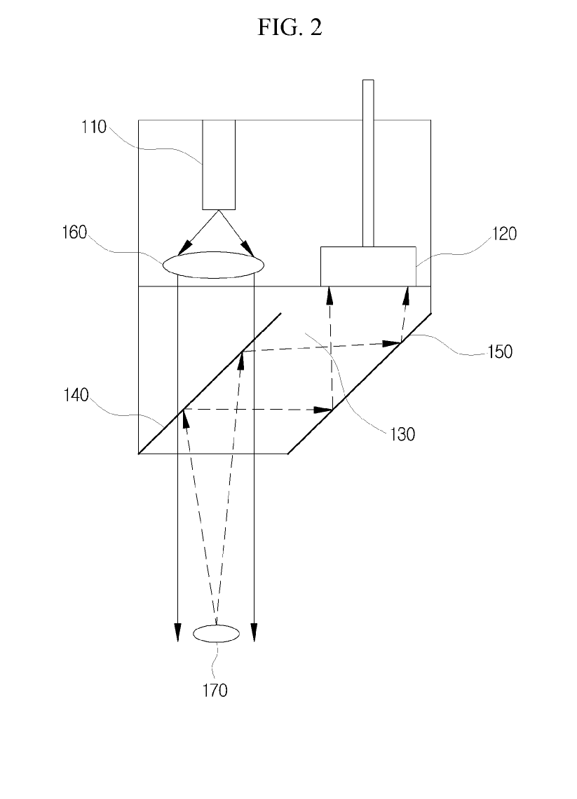

FIG. 2 is a view illustrating a structure of a photo-acoustic probe according to a first embodiment of the present invention.

Referring to FIG. 2, the photo-acoustic probe includes a laser generator 110, an ultrasound transducer 120, a medium material 130, first and second reflectors 140 and 150, a lens and beam controller 160.

A laser beam output from the laser generator 110 transmits through the first reflector 140 and is incident to an object 170.

The ultrasound transducer 120 is positioned in parallel on one side of the laser generator 110. Ultrasound generated by the object 170 is reflected from the first and second reflectors 140 and 150, and a reflected ultrasound beam is received by the ultrasound transducer 120 and the ultrasound transducer 120 analyzes the ultrasound information.

In order to minimize loss during ultrasound reflection and laser transmission, the photo-acoustic probe is filled with the medium material 130. Here, the medium material 140 may include a frame having an inner space and a liquid accommodated in the inner space of the frame.

A liquid, the medium material 130, may be a material having acoustic impedance similar to that of the object 170.

Also, the liquid, the medium material 130, may be a material having a low absorption coefficient of ultrasound generated by the object 170.

In order to reduce optical loss, the first and second reflectors 140 and 150 may be formed of a thin glass plate (slide glass) as a material having a refractive index similar to that of the medium material 130 and having acoustic impedance significantly different from that of a sound wave from the object 170.

The first reflector 140 may allow a laser beam output from the laser generator 110 to be transmitted therethrough and incident to the object 170.

The first reflector 140 is positioned to face the object 170 and reflects ultrasound generated by the object 170 toward the second reflector 150.

The second reflector 150 is positioned to face the ultrasound transducer 120 and reflects ultrasound reflected by the first reflector 140 toward the ultrasound transducer 120.

The lens and beam controller 160 is positioned between the layer generator 110 and the medium material 130 and controls a laser beam to produce optimal conditions for generating a photo-acoustic signal.

Thus, a laser beam output from the laser generator 110 passes through the lens and beam controller 160 and transmits through the medium material 130 so as to be irradiated to the object 170.

Ultrasound generated by the object 170 passes through the medium material 130 and is incident to the ultrasound transducer 120, and the receiver 120 receives the incident ultrasound and analyzes ultrasound information. Here, the ultrasound generated by the object 170 is reflected by the first reflector 140, passes through the medium material 130, is reflected by the second reflector 150, and is subsequently incident to the ultrasound transducer 120.

Any material other than the medium material 130 may be used as a transmission medium of ultrasound; however, since a liquid has a low sound wave transmission coefficient, loss of ultrasound may be reduced.

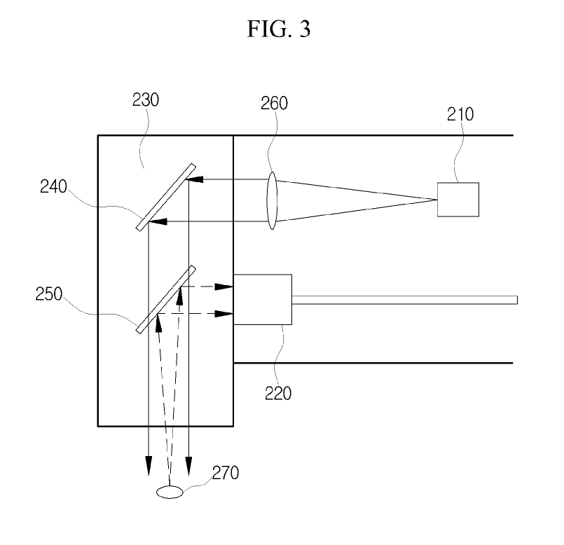

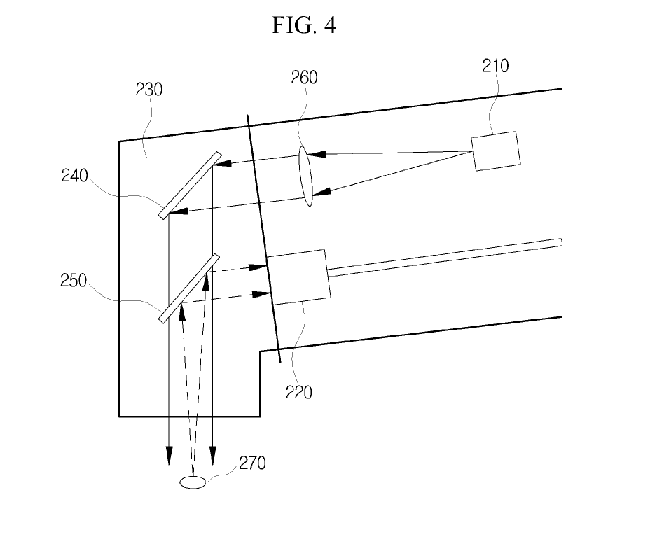

FIG. 3 is a view illustrating a structure of a photo-acoustic probe according to a second embodiment of the present invention, and FIG. 4 is a view illustrating a modified structure of the photo-acoustic probe illustrated in FIG. 3.

Components and functions of the components illustrated in FIGS. 3 and 4 are identical, and thus, the components illustrated in FIG. 3 will be mainly described.

Referring to FIG. 3, a photo-acoustic probe according to the second embodiment of the present invention includes a laser generator 210, an ultrasound transducer 220, a medium material 230, third and fourth reflectors 240 and 250, a lens and beam controller 260.

The laser generator 210 outputs a laser beam to an object 270, and the laser beam output from the laser generator 210 is incident to the medium material 230 and incident to the object 270 by the third and fourth reflectors 240 and 250 within the medium material 230.

The ultrasound transducer 220 is positioned in parallel on one side of the laser generator 210, receives ultrasound generated by the object 270, and analyzes ultrasound information.

The medium material 230 may be positioned on one side of the laser generator 210 and the ultrasound transducer 220 to form a movement path of the laser beam and ultrasound and provide a space in which the third and fourth reflectors 240 and 250 are disposed. In this case, the medium material 230 may include a frame having an inner space and a liquid accommodated in the inner space of the frame.

A liquid, the medium material 230, may be a material having acoustic impedance similar to that of ultrasound generated by the object 270.

Also, the liquid, the medium material 230, may be a material having a low absorption coefficient of ultrasound generated by the object 270 and light generated by the laser generator 210.

The third and fourth reflectors 240 and 250 may be positioned within the medium material 230 and change a movement path of the laser beam and ultrasound. One reflector changes a movement path of the laser beam, and the other reflector changes a movement path of ultrasound.

In the present embodiment, it is assumed that the third reflector 240 is disposed in parallel to the laser generator 210 to change a movement path of a laser beam and the fourth reflector 250 is disposed to face the ultrasound transducer 220 to change a movement path of ultrasound.

The third reflector 240 reflects a laser beam to the object 270, and the fourth reflector 250 allows the laser beam to be transmitted therethrough and reflects the ultrasound to the ultrasound transducer 220. The third reflector 240 may be configured to totally reflect the laser beam and the fourth reflector 250 may be configured to totally reflect the ultrasound.

The third reflector 240 may be formed of a material having high optical reflectivity, and may be formed of a mirror or a prism to increase optical reflectivity.

The fourth reflector 250 may be formed of a material having acoustic impedance significantly different from that of a liquid as the medium material 230 and the object 270.

The fourth reflector 250 may be formed of slide glass to reduce optical loss.

The lens and beam controller 260 may be positioned between the laser generator 210 and the medium material 230 to control a laser beam output from the laser generator 210.

In FIG. 3, the laser generator 210 and the ultrasound transducer 220 are disposed to be perpendicular to the laser beam incident to the object 270.

In comparison, as illustrated in FIG. 4, the laser generator 210 and the ultrasound transducer 220 may be disposed at a predetermined obtuse angle with respect to the laser beam incident to the object 270. Here, the third and fourth reflectors 240 and 250 are appropriately disposed to allow the laser beam to be incident to the object 270 and the ultrasound to be incident to the ultrasound transducer 220 according to positions of the laser generator 210 and the ultrasound transducer 220.

According to embodiments of the present invention, in the structure of a photo-acoustic probe for a clinical image, since an ultrasound beam positioned to be coaxial with a laser beam output from the laser generator is measured by the ultrasound transducer, a dark zone may be reduced.

Ultrasound transmission loss, which may be made in the structure of a photo-acoustic probe for a clinical image in which the axes of the laser generator and the ultrasound transducer are parallel, may be minimized

The photo-acoustic probe for a clinical image has been described according to the embodiments, but the scope of the present invention is not limited to a specific embodiment. The present invention may be corrected and modified within the technical scope obvious to those skilled in the art.

A number of exemplary embodiments have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A photo-acoustic probe for a clinical image, the photo-acoustic probe comprising:
a laser generator configured to generate a laser beam;
an ultrasound transducer disposed to be parallel to the laser generator and configured to analyze ultrasound output from an object;
first and second reflectors configured to receive ultrasound generated in an axis identical to that of the laser beam generated by the laser generator; and
a medium material configured to allow the laser to be transmitted from the first reflector to the object and increase ultrasound reflectivity of the first and the second reflector.

2. The photo-acoustic probe of claim 1, wherein the medium material includes a frame having an inner space and a liquid accommodated in the inner space.

3. The photo-acoustic probe of claim 1, wherein the first and second reflectors are formed of a glass plate (slide glass).

4. The photo-acoustic probe of claim 1, further comprising a lens and beam controller positioned between the laser generator and the medium material to control a laser beam generated by the laser generator.

5. A photo-acoustic probe for a clinical image, the photo-acoustic probe comprising:
a laser generator configured to generate a laser beam;
an ultrasound transducer disposed to be parallel to the laser generator and configured to analyze ultrasound output from an object;
a first reflector configured to reflect the laser beam so as to be incident to the object;
a second reflector configured to allow the laser beam reflected from the first reflector to be transmitted therethrough and reflect the ultrasound so as to be incident to the ultrasound transducer; and
a medium material configured to transmit the laser beam to the object and transmit the generated ultrasound to the ultrasound transducer.

6. The photo-acoustic probe of claim 5, wherein the medium material includes a frame having an inner space and a liquid accommodated in the inner space.

7. The photo-acoustic probe of claim 5, wherein the first reflector includes a mirror or a prism.

8. The photo-acoustic probe of claim 5, wherein the second reflector includes a glass plate (slide glass).

9. The photo-acoustic probe of claim 5, wherein the second reflector allows the laser beam to be transmitted therethrough and totally reflects the ultrasound.

10. The photo-acoustic probe of claim 5, further comprising a lens and beam controller positioned between the laser generator and the medium material to control a laser beam generated by the laser generator.

11. The photo-acoustic probe of claim 5, wherein the laser generator and the ultrasound transducer is disposed to be perpendicular to the laser beam incident to the object.

12. The photo-acoustic probe of claim 8, wherein the laser generator and the ultrasound transducer is disposed at a predetermined angle with respect to the laser beam incident to the object, rather than being perpendicular to the laser beam.
